(19) United States
(12) Patent Application Publication
(10) Pub. No.: US 2014/0135472 A1
(43) Pub. Date: May 15, 2014

(54) AFFINITY TAGS ABLE TO DETECT FRAMESHIFTS

(71) Applicant: Syndecion, LLC, Highland, MD (US)

(72) Inventors: Alan D. King, Highland, MD (US); Stephen B. Deitz, Catonsville, MD (US)

(73) Assignee: Syndecion, LLC, Highland, MD (US)

(21) Appl. No.: 14/070,648

(22) Filed: Nov. 4, 2013

Related U.S. Application Data
(60) Provisional application No. 61/722,536, filed on Nov. 5, 2012.

(51) Int. Cl. C07K 2/00

(52) U.S. Cl. CPC C07K 2/00
USPC 530/300; 536/23.1

(57) ABSTRACT

Described are polynucleotide sequences that encode peptide affinity tags with the surprising property that all of the possible out-of-frame peptide sequences are identical to each other, yet are distinct from the in-frame encoded peptide tag. The present invention includes peptide affinity tags incorporated in expressed proteins. Also described are ligands and antibodies that recognize the in-frame peptides and separate ligands or antibodies that recognize the out-of-frame peptides.

### CROSS-REFERENCES TO RELATED APPLICATIONS

This application claims the priority benefit under 35 U.S.C. §119(e) of U.S. Provisional Application Ser. No. 61/722,536, entitled “AFFINITY TAGS ABLE TO DETECT FRAMESHIFTS” filed Nov. 5, 2012, which is incorporated herein by reference in its entirety.

### BACKGROUND OF THE INVENTION

1. Field of the Invention

The present invention relates to the field of biotechnology and more specifically to the artificial sequences used as affinity tags biotechnological applications.

2. Background of the Prior Art

Peptide affinity ligands are useful for several processes. One important use is for diagnostic or research assays. Antibodies or ligands that recognize a specific assay target can be labeled with a peptide affinity tag and identified by having a second ligand that recognizes the affinity tag and also is labeled with an enzyme or other means to produce a detectable signal.

A number of natural and synthetic peptide affinity tags are known to the industry and have been used for affinity purification and diagnostic assays. None of the known peptide affinity tags have the property of the present invention wherein amino acids expressed from their nucleotide sequences have the same amino acid sequence when the nucleotide sequence is out-of-frame in either +1 or −1 direction. It would be desirable to be able to detect amino acids sequences produced by out-of-frame gene expression using a single affinity ligand that recognizes the out of frame sequence and differs from the ligand that recognizes the in-frame sequence.

### SUMMARY OF THE INVENTION

Described are polynucleotide sequences that encode peptide affinity tags with the surprising property that all of the possible out-of-frame peptide sequences are identical to each other, yet are distinct from the in-frame encoded peptide tag. The present invention includes peptide affinity tags incorporated in expressed proteins. Also described are ligands and antibodies that recognize the in-frame peptides and separate ligands or antibodies that recognize the out-of-frame peptides.

The present invention includes nucleotide sequences that encode one amino acid sequence when expressed in-frame and different amino acid sequences when expressed out-of-frame, wherein, said out-of-frame amino acid sequences are identical if the out-of-frame mutation is either a +1 or a −1 nucleotide mutation. The present invention also includes peptide sequences produced by the expression of nucleotide sequences that encode one amino acid sequence when expressed in-frame and different amino acid sequences when expressed out-of-frame, wherein, said out-of-frame amino acid sequences are identical if the out-of-frame mutation is either a +1 or a −1 nucleotide mutation.

A −1 frameshift refers to a deletion mutation that results in a shift in reading frame by loss of one nucleotide and a +1 frameshift refers to an insertion mutation that results in a shift in reading frame by +1 nucleotide. Deletion mutations that result in a shift in reading frame of two nucleotides are in the same reading frame as +1 frameshifts. Insertion mutations that result in a shift in reading frame of two nucleotides are in the same reading frame as −1 frameshifts. Either of these mutations occur in sequences prior to the affinity tag sequence.

One form of the invention includes a nucleotide sequence that encodes one amino acid sequence when expressed in-frame and a different amino acid sequence when expressed out-of-frame as a result of a single nucleotide change, wherein said out-of-frame amino acid sequence is the same whether single nucleotide change is a nucleotide addition or deletion. This can comprise at least two repeats of the nucleotide sequence ctttcc (example SEQ ID No. 1, 9, 17, 25, 34, 42), at least two repeats of the nucleotide sequence tccctt (example SEQ ID No. 2, 10, 18, 26, 35, 43, 116), at least two repeats of the nucleotide sequence agggaa (example SEQ ID No. 3, 11, 19, 27, 36, 44), or at least two repeats of the nucleotide sequence gaaagg (example SEQ ID No. 4, 12, 20, 28, 29, 37, 45). A preferred embodiment would have 2-10 repeats of a nucleotide sequence selected from the list; gaaagg, cttcc, tcctt, or agggaa.

Another aspect of the invention is ligands, to include antibodies and antibody fragments, that bind to amino acid sequences of the present invention. In this form of the invention a ligand that specifically binds to an amino acid sequence that comprises two or more repeats of amino acid pairs selected from the lists of in-frame amino acid pairs leucine-serine (example SEQ ID No. 30, 58, 90); serine-leucine (example SEQ ID No. 31, 59, 75, 91); arginine-glutamic acid (example SEQ ID No. 32, 60, 76, 92); glutamic acid-arginine (example SEQ ID No. 33, 61, 77, 93) or of out-of-frame amino acid pairs proline-phenylalanine (example SEQ ID No. 55, 71, 87, 103); phenylalanine-proline (example SEQ ID No. 54, 70, 86, 102); lysine-glycine (example SEQ ID No. 57, 73, 89, 105); glycine-lysine (example SEQ ID No. 56, 72, 88, 104, 131). These amino acid sequences can have an additional amino acid in the form of in-frame amino acid pairs (leucine-serine)n+leucine (example SEQ ID No. 66, 74, 82, 98); (serine-leucine)n+serine (example SEQ ID No. 67, 83, 99); (arginine-glutamic acid)n+arginine (example SEQ ID No. 68, 84, 100); (glutamic acid-arginine)n+glutamic acid (example SEQ ID No. 69, 101, 127) or out-of-frame amino acid pairs (proline-phenylalanine)n+proline (example SEQ ID No. 51, 63, 79, 95); (phenylalanine-proline)n+phenylalanine (example SEQ ID No. 50, 62, 78, 94); (lysine-glycine)n+lysine (example SEQ ID No. 53, 65, 81, 85, 97); or (glycine-lysine)n+glycine (example SEQ ID No. 52, 64, 80, 96) wherein n represents n number of amino acid pairs and wherein n is 2 or greater.

In one aspect of the invention, amino acid sequences of the present invention are amino acid sequences that are comprised of repeats of the amino acid pairs proline-phenylalanine; phenylalanine-proline; lysine-glycine; or glycine-lysine.

In another aspect of the invention, amino acid sequences of the present invention comprise two or more repeats of amino acid pairs selected from the list comprising in-frame amino acid pairs leucine-serine (example SEQ ID No. 30, 58, 90); serine-leucine (example SEQ ID No. 31, 59, 75, 91); arginine-glutamic acid (example SEQ ID No. 32, 60, 76, 92); glutamic acid-arginine (example SEQ ID No. 33, 61, 77, 93) or out-of-frame amino acid pairs proline-phenylalanine (example SEQ ID No. 55, 71, 87, 103); phenylalanine-proline (example SEQ ID No. 54, 70, 86, 102); lysine-glycine (example SEQ ID No. 57, 73, 89, 105); glycine-lysine (example SEQ ID No. 56, 72, 88, 104, 131). These sequences can additional an amino acid in the form of in-frame amino acid pairs (leucine-serine)n+leucine (example SEQ ID No. 66, 74, 82, 98); (serine-leucine)n+serine (example SEQ ID No. 67, 83, 99); (arginine-glutamic acid)n+arginine (example SEQ ID No. 68, 84, 100); (glutamic acid-arginine)n+glutamic acid (example SEQ ID No. 69, 101, 127) or out-of-frame amino acid pairs (proline-phenylalanine)n+proline (example SEQ ID No. 51, 63, 79, 95); (phenylalanine-proline)n+phenylalanine (example SEQ ID No. 50, 62, 78, 94); (lysine-glycine)n+lysine (example SEQ ID No. 53, 65, 81, 85, 97); or (glycine-lysine)n+glycine (example SEQ ID No. 52, 64, 80, 96) wherein n represents n number of amino acid pairs and wherein n is 2 or greater and the total number of amino acids in the sequence is an odd number.

### BRIEF DESCRIPTION OF THE DRAWING

FIG. 1 is a table showing codon pairs of embodiments of the invention and the amino acids that they encode.

FIG. 2 is an example of a prior art polynucleotide sequence expressing a different amino acid sequence in each of the frames. The gene sequence is from a human antibody light chain (gene bank JX206997.1) (SEQ IN No 110). The in-frame amino acid sequence (SEQ ID No 111), the insertion frameshift amino acid sequence (SEQ ID No 112) and the deletion frameshift amino acid sequence (SEQ ID 113) are all different. This is an example of an average sequence that is not the present invention.

FIG. 3 is an example of an embodiment of the present invention showing a polynucleotide sequence that is different for all three expression frames (SEQ ID No 25, 114 and 115), but the amino acid sequence produced by expression after a +1 insertion frameshift of the polynucleotide sequence (SEQ ID No 50) is identical to the amino acid sequence produced by a −1 deletion frameshift of the same polynucleotide sequence (SEQ ID No 50).

FIG. 4 is another example of an embodiment of the present invention showing polynucleotide sequences that are different for all three expression frames (SEQ ID No 116, 117, 118), but the amino acid sequence (SEQ ID No 51) produced by expression of a +1 insertion frameshift of the polynucleotide sequence is identical to the amino acid sequence (SEQ ID No 51) produced by expression of a −1 deletion frameshift of the same polynucleotide sequence.

FIG. 5 is another example of an embodiment of the present invention showing polynucleotide sequences that are different for all three expression frames (SEQ ID No 27, 119, 120), but the amino acid sequence (SEQ ID No 52) produced by expression of a +1 insertion frameshift of the polynucleotide sequence is identical to the amino acid sequence (SEQ ID No 52) produced by expression of a −1 deletion frameshift of the same polynucleotide sequence.

FIG. 6 is another example of an embodiment of the present invention showing polynucleotide sequences that are different for all three expression frames (SEQ ID No 28, 121, 122), but the amino acid sequence (SEQ ID No 53) produced by expression of a +1 insertion frameshift of the polynucleotide sequence is identical to the amino acid sequence (SEQ ID No 53) produced by expression of a −1 deletion frameshift of the same polynucleotide sequence.

FIG. 7 is an example of the present invention showing a polynucleotide sequence that is different for all three expression frames (SEQ ID No 33, 123, 124), but the amino acid sequence (SEQ ID No 87) produced by expression of a +1 insertion frameshift of the polynucleotide sequence is identical to the amino acid sequence (SEQ ID No 87) produced by expression of a −1 deletion frameshift of the same polynucleotide sequence.

FIG. 8 is another example of an embodiment of the present invention showing polynucleotide sequences that are different for all three expression frames (SEQ ID No 38, 125, 126), but the amino acid sequence (SEQ ID No 86) produced by expression of a +1 insertion frameshift of the polynucleotide sequence is identical to the amino acid sequence (SEQ ID No 86) produced by a −1 deletion frameshift of the same polynucleotide sequence.

FIG. 9 is another example of an embodiment of the present invention showing polynucleotide sequences that are different for all three expression frames (SEQ ID No 41, 128, 129), but the amino acid sequence (SEQ ID No 89) produced by expression of a +1 insertion frameshift of the polynucleotide sequence is identical to the amino acid sequence (SEQ ID No 89) produced by expression of a −1 deletion frameshift of the same polynucleotide sequence.

FIG. 10 is another example of an embodiment of the present invention showing polynucleotide sequences that are different for all three expression frames (SEQ ID No 40, 130, 132), but the amino acid sequence (SEQ ID No 131) produced by expression of a +1 insertion frameshift of the polynucleotide sequence is identical to the amino acid sequence (SEQ ID No 131) produced by expression of a −1 deletion frameshift of the same polynucleotide sequence.

FIG. 11 is another example of an embodiment of the present invention showing in-frame DNA sequence (SEQ ID No 133), in-frame amino acid sequence (SEQ ID No 134) and out-of frame amino sequence (SEQ ID No 135) of this embodiment of the present invention connected to a linker that enhances aspects of the invention at the linker connection.

FIG. 12 is another example of an embodiment of the present invention showing in-frame DNA sequence (SEQ ID No 136), in-frame amino acid sequence (SEQ ID No 137) and out-of frame amino sequences (SEQ ID No 138, 139) of this embodiment of the present invention connected to a linker that link without enhancing aspects of the invention at the linker connection.

### DETAILED DESCRIPTION

Peptide affinity tags are amino acid sequences inserted in a protein to assist detection or purification of that protein. Recombinant proteins produced in a fermenter are produced among a mixture of other proteins. It is important to be able to economically recover the desired proteins with high purity from the complicated mix from which it resides. Affinity purification is one method. In order to eliminate the need to produce antibodies or ligands that specifically bind to every different protein produced, affinity tags can be added to an amino acid sequence. These tags are encoded in DNA in genetic sequences used to express the proteins and they produce amino acid sequences that can be recognized by specific ligands or antibodies. Such affinity tags are amino acid sequences that are inserted into a polypeptide or protein to assist detection or purification of that polypeptide or protein. Affinity tags are typically inserted into polypeptides or proteins by manipulating the encoding polynucleotides for those polypeptides or proteins. Affinity tags are used for identifying proteins in assays; they can be used for purification of proteins and have other uses.

Artificial affinity tags described here have the surprising property of being expressed as one amino acid sequence when expressed in-frame from their encoding polynucleotide sequences and a distinct separate and unique amino acid sequence when expressed out-of-frame with the additional property that the peptide expressed from an out-of frame nucleotide sequence is the same whether the frameshift consists of a gain (+1) or a loss (−1) of a nucleotide. This occurs despite the fact that the polynucleotide sequences in the +1 and −1 frameshift may differ. In fact, this is another surprising aspect of one embodiment of the present invention.

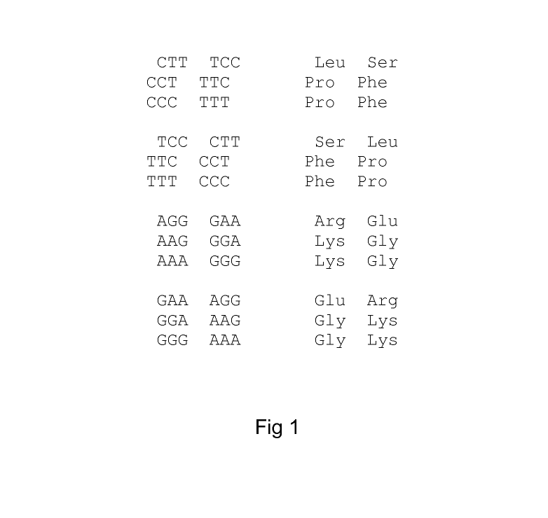

Nucleotide sequences that produce identical amino acid sequences in each out of frame direction do not produce identical out of frame nucleotide codons. They have alternate codon usage in each direction. FIG. 1 shows four groups of codon sets. The first line in each set is the in-frame codons. The next two lines of each set show the out-of-frame codons. It is apparent from inspection of this table that the two out-of frame codon pairs differ in the two out-of-frame directions. Thus it is not obvious from a visual inspection of a codon table that the deletion or addition of a single nucleotide as disclosed in one embodiment of the present invention would result in the same amino acid sequence whether there is a deletion or addition so the single amino acid.

One aspect of another embodiment of the invention comprises ligands and antibodies that recognize the in-frame peptide and separate ligands or antibodies that recognize the out-of-frame peptides. As utilized herein, antibodies, antibody fragments, single domain antibodies, single chain antibodies and other forms or fragments of antibodies are a subset of the term ligand.

Candidate codon combinations and corresponding amino acids with the desired properties are shown in FIG. 1.

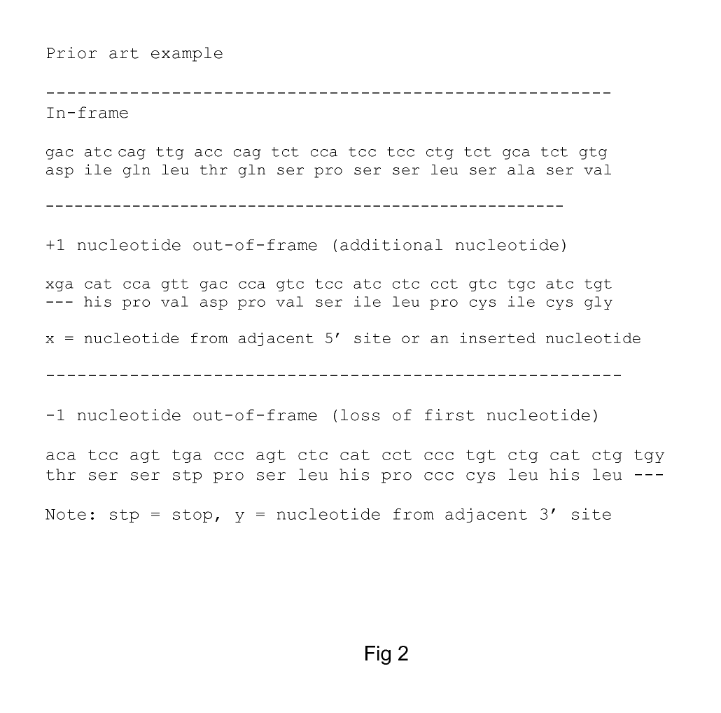

FIG. 2 shows a prior art sequence that does not have the characteristics of the present invention. Each out-of-frame sequence is different.

When mentioned, a −1 frameshift refers to a deletion mutation that results in a shift in reading frame by −1 nucleotide and a +1 frameshift refers to an insertion mutation that results in a shift in reading frame by +1 nucleotide. Deletion mutations that result in a shift in reading frame of two nucleotides are in the same reading frame as +1 frameshifts. Insertion mutations that result in a shift in reading frame of two nucleotides are in the same reading frame as −1 frameshifts.

In one aspect of the invention, multiple codon (trinucleotide) pairs are used in genetic sequences to express alternating amino acids. Codon pairs can be selected from the list comprising ctttcc, tccctt, agggaa or gaaagg. The codon pairs are used as multiples in the form of (ctttcc)×n, (tccctt)×n, (agggaa)×n, or (gaaagg)×n where n is any number greater than 1. For example, (ctttcc)×2 is ctttccctttcc (SEQ ID No. 1), (tccctt)×2 is tccctttccctt (SEQ ID No. 2), agggaa×2 is agggaaagggaa (SEQ ID No. 3) and gaaagg×2 is gaaagggaaagg (SEQ ID No. 4). They can also be used in codon pair multiples with a preceding or following single codon to make an odd number of codons. This can take the form of ((ctttcc)×n)+ctt, ((tccctt)×n)+tcc, ((agggaa)×n)+agg, or ((gaaagg)×n)+gaa, where “n” is any number greater than 1. For example, ((ctttcc)×2)+ctt is ctttccctttccctt (SEQ ID No. 5), ((tccctt)×2)+tcc is tccctttccctttcc (SEQ ID No. 6), (agggaa×2)+agg is agggaaagggaaagg (SEQ ID No. 7) and (gaaagg×2)+gaa is gaaagggaaagggaa (SEQ ID No. 8). They also can take the form of tcc+((ctttcc)×n), ctt+((tccctt)×n), gaa+((agggaa)×n) or agg+((gaaagg)×n) where n is any number greater than 1. For example, tcc+((ctttcc)×2) is tccctttccctttcc (SEQ ID No. 6), ctt+((tccctt)×2) is ctttccctttccctt (SEQ ID No. 5), gaa+(agggaa×2) is gaa+agggaaagggaa (SEQ ID No. 8) and agg+(gaaagg×2) is agggaaagggaaagg (SEQ ID No. 7). In a preferred mode n is from 2 to 12.

Using different formula nomenclature, the codon pairs are used as multiples in the form of (ctttcc)n, (tccctt)n, (agggaa)n or (gaaagg)n where n is any number greater than 1 and ( )n indicates multiplication. They also can be used in codon pair multiples with a preceding or following single codon to make an odd number of codons. This can take the form of [(ctttcc)n+ctt], [(tccctt)n+tcc], [(agggaa)n+agg], [(gaaagg)n+gaa], [tcc+(ctttcc)n], [ctt+(tccctt)n], [gaa+(agggaa)n] or [agg+(gaaagg)n].

Other examples of codon combinations corresponding to the formulas in the preceding two paragraphs are illustrated in sequences shown in (SEQ ID No. 9) through (SEQ ID No. 24). This is not an exhaustive sequence list.

Genetic manipulations such as the polymerase chain reaction can introduce mutations in a nucleotide sequence. Especially disruptive to expressed protein sequences are frameshift mutations that change the expressed amino acid sequence beyond the frameshift. This can lead to loss of product and/or contamination of product. The ability to detect and/or remove contaminating proteins from protein mixtures can be useful. In most cases, frameshift mutations lead to amino acid sequences that are different in all three frames. This includes −1 and +1 frameshifts which are not the same. As an example, the gene sequence (SEQ ID No. 110) in FIG. 2 is taken from a human antibody light chain (gene bank JX206997.1). In this prior art example, in-frame and −1 and +1 frameshift mutations for this sequence result in three completely different amino acid sequences downstream of each in-frame expression and frameshift mutation ((SEQ ID No. 110), (SEQ ID No. 112) and (SEQ ID No. 113) respectively. In other words, amino acid sequences resulting from −1 mutations are not the same as amino acid sequences resulting from +1 mutations. In all examples, nucleotide frameshift mutations must occur prior to the expressed sequence. This is an example of amino acid sequences resulting from nucleotide frameshifts that are not of the invention.

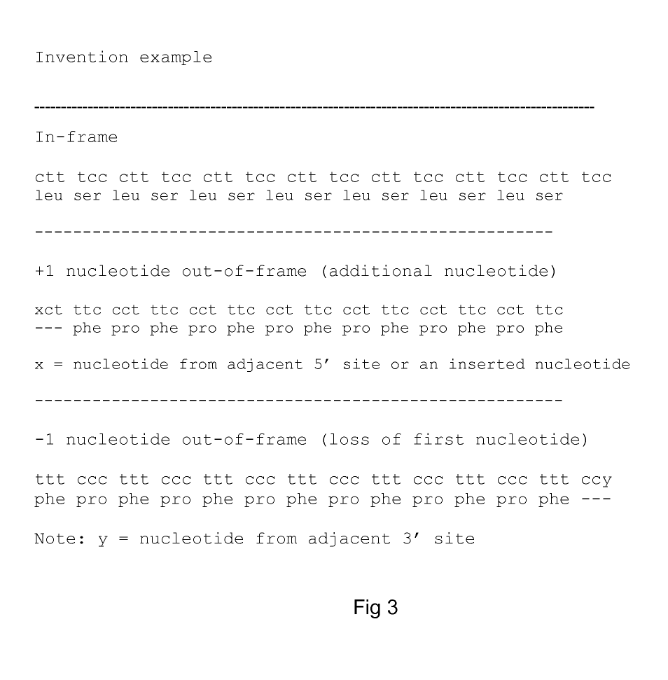

Nucleotide sequences of the invention encode one amino acid sequence when in-frame and another amino sequence when out-of-frame. But surprisingly, in contrast to the previous example, each out-of-frame nucleotide sequence encodes the same amino acid sequence if a nucleotide is gained (+1) or if one is lost (−1). FIGS. 3 through 12 illustrate this phenomenon.

One aspect of the invention would be polynucleotide sequences of the invention encoding an even number of in-frame amino acids, resulting in an odd number of out-of frame amino acids (if there were a frameshift). These would take the form of [(ctttcc)×n]; [(tccctt)×n]; [(agggaa)×n]; or [(gaaagg)×n], or equivalently, [(ctttcc)n]; [(tccctt)n]; [(agggaa)n]; or [(gaaagg)n], where “n” is any integer greater than 1. Typical ranges for “n” are 2 to 8. In another aspect of the invention, ranges for “n” are 2 to 12. FIGS. 3 through 6 illustrate this.

FIG. 3 is an example of the present invention showing a polynucleotide sequence that is different for all three expression frames, but the amino acid sequence produced by expression after a +1 insertion frameshift of the polynucleotide sequence is identical to the amino acid sequence produced by a −1 deletion frameshift of the same polynucleotide sequence. The reading frame of a +2 insertion would be identical to the reading frame of a −1 deletion; the reading frame of a −2 deletion would be identical to the reading frame of a +1 insertion. The amino acid sequence produced by in-frame expression differs from that produced by out-of frame expression. Thus it is possible to differentiate between in-frame and out-of-frame expression using only two ligands or antibodies that recognize the two distinct peptides that are produced by in-frame and out-of-frame expression. In this example, an even number of trinucleotide codons produces an odd number of amino acids in each out-of-frame expression.

In one aspect of the invention, FIG. 3 shows a nucleotide sequence ctt tcc ctt tcc ctt tcc ctt tcc ctt tcc ctt tcc ctt tcc (SEQ ID No. 25) which is 7 repetitions of the codon pair ctt tcc. This sequence results in expression of the amino acid sequence leu ser leu ser leu ser leu ser leu ser leu ser leu ser (SEQ ID No. 30). A +1 nucleotide out-of-frame mutation (additional preceding nucleotide) gives a sequence of nct ttc cct ttc cct ttc cct ttc cct ttc cct ttc cct ttc (SEQ ID No. 114)) resulting in an amino acid sequence of - - - phe pro phe pro phe pro phe pro phe pro phe pro phe (SEQ ID No. 50). A −1 nucleotide out-of-frame mutation (loss of preceding nucleotide) gives a sequence of ttt ccc ttt ccc ttt ccc ttt ccc ttt ccc ttt ccc ttt ccy (SEQ ID No. 115) resulting in an amino acid sequence of phe pro phe pro phe pro phe pro phe pro phe pro phe - - - (SEQ ID No. 50). This is identical to the sequence expressed by the +1 mutation. (Note that n=a nucleotide from the adjacent 5′ site [or an inserted nucleotide] and y=a nucleotide from the adjacent 3′ site). The amino acid sequence is the same with each frameshift even though the nucleotide sequence differs.

It is recognized in the examples, that each of the out-of-frame sequences are one codon shorter than the in-frame sequence of the invention and the that loss of a codon occurs at different ends of the sequence for +1 and −1 frameshift mutations. For the +1 frameshift, the loss is at the 5′ end of the selected sequence and, for the −1 frameshift, the loss is at the 3′ end of the selected sequence. In spite of that, both encoded amino acid sequences are the same.

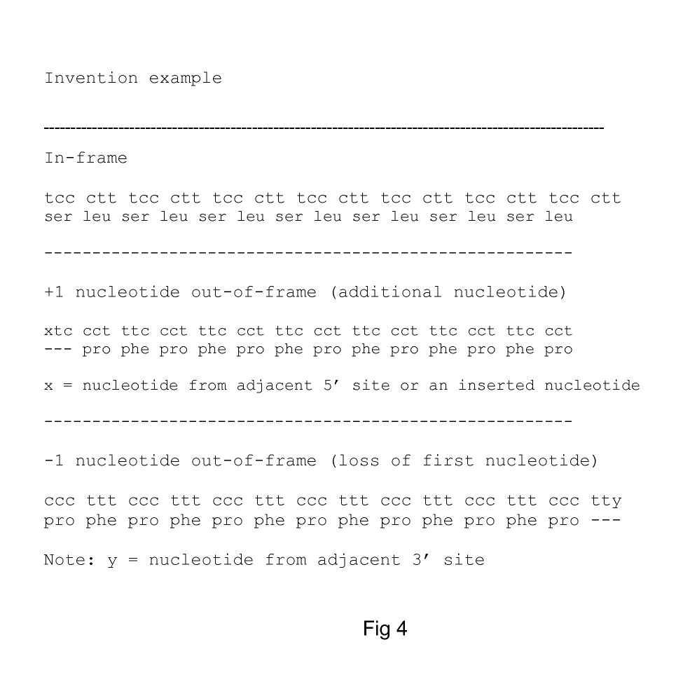

FIG. 4 is another example of the present invention showing a polynucleotide sequence that is different for all three expression frames, but the amino acid sequence produced by expression of a +1 insertion frameshift of the polynucleotide sequence is identical to the amino acid sequence produced by expression of a −1 deletion frameshift of the same polynucleotide sequence. The reading frame of a +2 insertion would be identical to the reading frame of a −1 deletion; the reading frame of a −2 deletion would be identical to the reading frame of a +1 insertion. In this case, the codons express the same amino acid pairs shown in FIG. 3 with the order reversed. The amino acid sequence produced by in-frame expression differs from that produced by out-of frame expression. Thus it is possible to differentiate between in-frame and out-of-frame expression using only two ligands or antibodies that recognize the two distinct peptides that are produced by in-frame and out-of-frame expression. In this example, an even number of trinucleotide codons produces an odd number of amino acids in each out-of-frame expression.

FIG. 4 shows a nucleotide sequence of tcc ctt tcc ctt tcc ctt tcc ctt tcc ctt tcc ctt tcc ctt (SEQ ID No. 116) which is 7 pairs of the codons tcc ctt. These codons express ser and leu (in the opposite order than shown in FIG. 3). This sequence results in expression of the amino acid sequence ser leu ser leu ser leu ser leu ser leu ser leu ser leu (SEQ ID No. 31). A +1 nucleotide out-of-frame mutation (additional preceding nucleotide) gives a sequence of ntc cct ttc cct ttc cct ttc cct ttc cct ttc cct ttc cct (SEQ ID No. 117) resulting in an amino acid sequence of - - - pro phe pro phe pro phe pro phe pro phe pro phe pro (SEQ ID No. 51). A −1 nucleotide out-of-frame mutation (loss of preceding nucleotide) gives a sequence of ccc ttt ccc ttt ccc ttt ccc ttt ccc ttt ccc ttt ccc tty (SEQ ID No. 118) resulting in an amino acid sequence of pro phe pro phe pro phe pro phe pro phe pro phe pro - - - (SEQ ID No. 51). This is identical to the amino acid sequence expressed by the +1 mutation. (Note that x=a nucleotide from the adjacent 5′ site [or an inserted nucleotide] and y=a nucleotide from the adjacent 3′ site). The amino acid sequence is the same with each frameshift even though the nucleotide sequence differs.

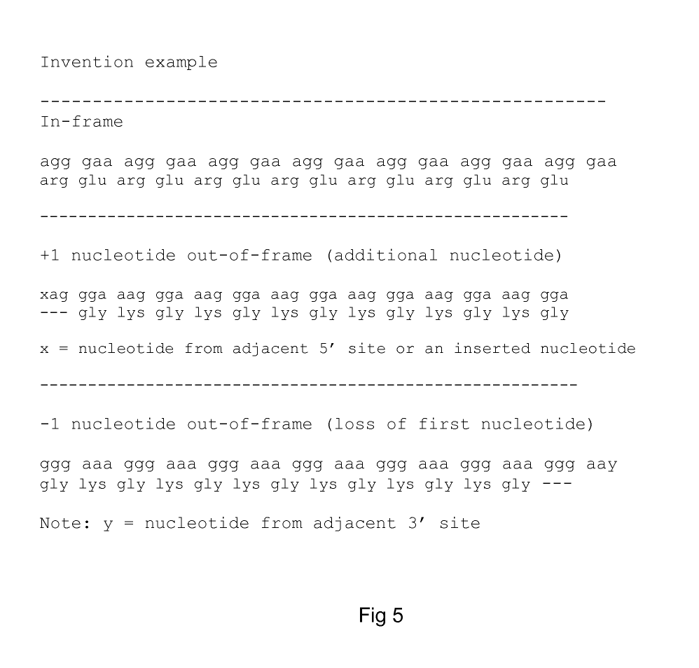

FIG. 5 is another example of an embodiment of the present invention showing polynucleotide sequences that are different for all three expression frames, but the amino acid sequence produced by expression of a +1 insertion frameshift of the polynucleotide sequence is identical to the amino acid sequence produced by expression of a −1 deletion frameshift of the same polynucleotide sequence. The reading frame of a +2 insertion would be identical to the reading frame of a −1 deletion; the reading frame of a −2 deletion would be identical to the reading frame of a +1 insertion. In this case, the codons express different amino acids than those shown in shown in FIGS. 3 and 4. The amino acid sequence produced by in-frame expression differs from that produced by out-of frame expression. Thus it is possible to differentiate between in-frame and out-of-frame expression using only two ligands or antibodies that recognize the two distinct peptides that are produced by in-frame and out-of-frame expression. In this example, an even number of trinucleotide codons produces an odd number of amino acids in each out-of-frame expression.

In the aspect of the invention shown in FIG. 5, agg gaa agg gaa agg gaa agg gaa agg gaa agg gaa agg gaa (SEQ ID No. 27) is 7 pairs of the codons agg gaa. These codons express arg and glu. This sequence results in expression of the amino acid sequence arg glu arg glu arg glu arg glu arg glu arg glu arg glu (SEQ ID No. 32). A +1 nucleotide out-of-frame mutation (additional preceding nucleotide) gives a sequence of nag gga aag gga aag gga aag gga aag gga aag gga aag gga (SEQ ID No. 119) resulting in an amino acid sequence of - - - gly lys gly lys gly lys gly lys gly lys gly lys gly (SEQ ID No. 52). A −1 nucleotide out-of-frame mutation (loss of preceding nucleotide) gives a sequence of ggg aaa ggg aaa ggg aaa ggg aaa ggg aaa ggg aaa ggg aay (SEQ ID No. 120) resulting in an amino acid sequence of gly lys gly lys gly lys gly lys gly lys gly lys gly - - - (SEQ ID No. 52). This is identical to the sequence expressed by the +1 mutation. (Note that n=a nucleotide from the adjacent 5′ site [or an inserted nucleotide] and y=a nucleotide from the adjacent 3′ site). The amino acid sequence is the same with each frameshift even though the nucleotide sequence differs.

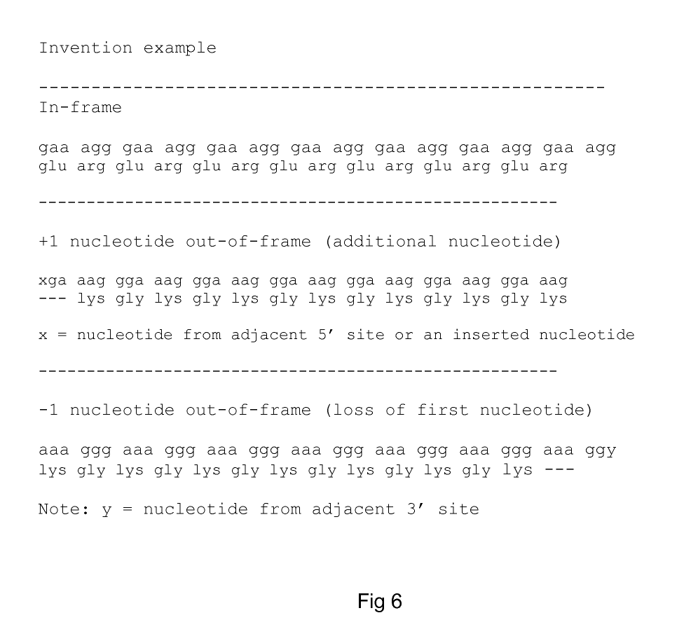

FIG. 6 is another example of an embodiment of the present invention showing polynucleotide sequences that are different for all three expression frames, but the amino acid sequence produced by expression of a +1 insertion frameshift of the polynucleotide sequence is identical to the amino acid sequence produced by expression of a −1 deletion frameshift of the same polynucleotide sequence. The reading frame of a +2 insertion would be identical to the reading frame of a −1 deletion; the reading frame of a −2 deletion would be identical to the reading frame of a +1 insertion. In this case, the codons express the same amino acid pairs shown in FIG. 5 with the order reversed. The amino acid sequence produced by in-frame expression differs from that produced by out-of frame expression. Thus it is possible to differentiate between in-frame and out-of-frame expression using only two ligands or antibodies that recognize the two distinct peptides that are produced by in-frame and out-of-frame expression. In this example, an even number of trinucleotide codons produces an odd number of amino acids in each out-of-frame expression.

The embodiment described in FIG. 6 shows a nucleotide sequence of gaa agg gaa agg gaa agg gaa agg gaa agg gaa agg gaa agg (SEQ ID No. 28) which is 7 pairs of the codons gaa agg. These codons express glu and arg (in the opposite order than shown in FIG. 5). This sequence results in expression of the amino acid sequence glu arg glu arg glu arg glu arg glu arg glu arg glu arg (SEQ ID No. 33). A +1 nucleotide out-of-frame mutation (additional preceding nucleotide) gives a sequence of nga aag gga aag gga aag gga aag gga aag gga aag gga aag (SEQ ID No. 121) resulting in an amino acid sequence of - - - lys gly lys gly lys gly lys gly lys gly lys gly lys (SEQ ID No. 53). A −1 nucleotide out-of-frame mutation (loss of preceding nucleotide) gives a sequence of aaa ggg aaa ggg aaa ggg aaa ggg aaa ggg aaa ggg aaa ggy (SEQ ID No. 122) resulting in an amino acid sequence of lys gly lys gly lys gly lys gly lys gly lys gly lys - - - . This is identical to the amino acid sequence expressed by the +1 mutation. (Note that n=a nucleotide from the adjacent 5′ site [or an inserted nucleotide] and y=a nucleotide from the adjacent 3′ site). The amino acid sequence is the same with each frameshift even though the nucleotide sequence differs.

Within each of the four above examples of the invention, the two out-of-frame nucleotide sequences are different, yet they produce an identical amino acid sequence. This is possible because of the redundancy of the genetic code. It makes discovery of in-frame nucleotide sequences that produce the same amino acid sequences with an out-of-frame nucleotide sequence difficult. No nucleotide sequences could be found that produced identical out-of-frame nucleotide sequences in the −1 and +1 directions.

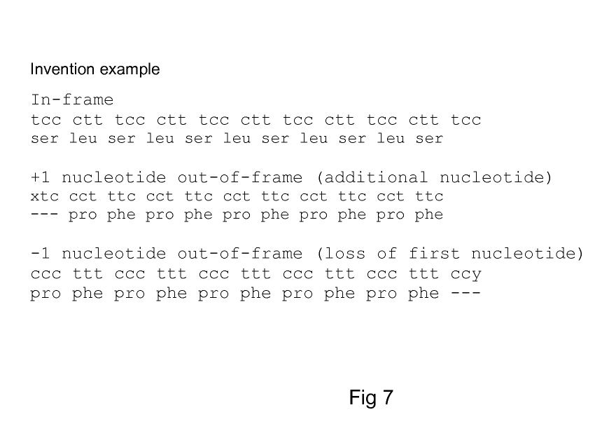

It also is apparent from the examples that the number of codons in a sequence of the invention is usually one less than the in-frame sequence after a one nucleotide frameshift (+1 or −1) has occurred. With an even number of codons in the in-frame sequence, this results in an odd number of codons in the out-of-frame sequences. In order to produce an out-of-frame sequence with an even number of codons, an odd number of codons must be used in the in-frame sequence. This may be achieved by adding half of a pair of codons (i.e. one codon) to one end of the in-frame sequence. Examples to illustrate this are shown in FIGS. 7-10.

Another aspect of the invention would be polynucleotide sequences of the invention encoding an odd number of in-frame amino acids, resulting in an even number of out-of frame amino acids if there were a frameshift. To reduce possible steric hindrance, multiple sets of amino acids may be used. 2-3 repeats of 5-15 amino acids could be used. These would take the form of [(ctttcc)×n]+ctt; [(tccctt)×n]+tcc; [(agggaa)×n]+agg; or [(gaaagg)×n]+gaa, or equivalently, [(ctttcc)n+ctt]; [(tccctt)n+tcc]; [(agggaa) n+agg]; or [(gaaagg)n+gaa], where “n” is any integer greater than 1. Typical ranges for “n” are 2 to 8. In another aspect of the invention, ranges for “n” are 2 to 12. FIGS. 7 through 10 illustrate this.

FIG. 7 shows a nucleotide sequence tcc ctt tcc ctt tcc ctt tcc ctt tcc ctt tcc (SEQ ID No. 33) which is 5 repetitions of the codon pair tcc cct with an extra tcc codon added to the 3′ end. This sequence results in expression of the amino acid sequence ser leu ser leu ser leu ser leu ser leu ser (SEQ ID No. 83). A +1 nucleotide out-of-frame mutation (additional preceding nucleotide) gives a sequence of ntc cct ttc cct ttc cct ttc cct ttc cct ttc (SEQ ID No. 123) resulting in an amino acid sequence of pro phe pro phe pro phe pro phe pro phe (SEQ ID No. 87). A −1 nucleotide out-of-frame mutation (loss of preceding nucleotide) gives a sequence of ccc ttt ccc ttt ccc ttt ccc ttt ccc ttt ccy (SEQ ID No. 124) resulting in an amino acid sequence of pro phe pro phe pro phe pro phe pro phe (SEQ ID No. 87). This is identical to the sequence expressed by the +1 mutation. The amino acid sequence is the same with each frameshift even though the nucleotide sequence differs. Both out-of-frame nucleotide sequences contain an even number of codons, in this example 10 codons, while the in-frame sequence has 11 codons.

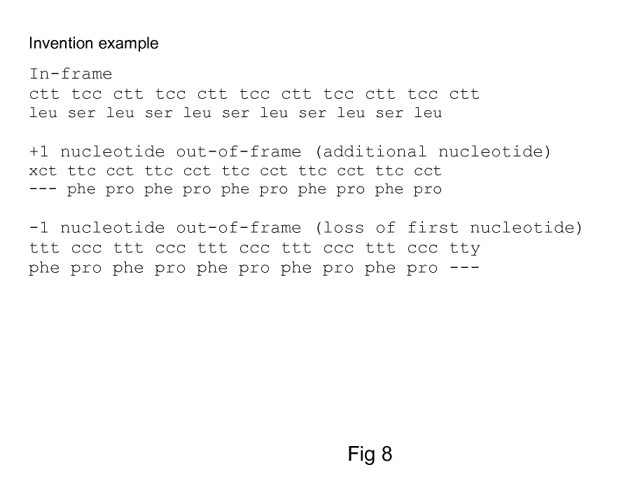

FIG. 8 shows a nucleotide sequence, ctt tcc ctt tcc ctt tcc ctt tcc ctt tcc ctt (SEQ ID No. 38) which is 5 pairs of the codons ctt tcc with an extra ctt codon added to the 3′ end. This sequence results in expression of the amino acid sequence leu ser leu ser leu ser leu ser leu ser leu (SEQ ID No. 82). A +1 nucleotide out-of-frame mutation (additional preceding nucleotide) gives a sequence of nct ttc cct ttc cct ttc cct ttc cct ttc ctt (SEQ ID No. 125) resulting in an amino acid sequence of phe pro phe pro phe pro phe pro phe pro (SEQ ID No. 86). A −1 nucleotide out-of-frame mutation (loss of preceding nucleotide) gives a sequence of ttt ccc ttt ccc ttt ccc ttt ccc ttt ccc tty (SEQ ID No. 126) resulting in an amino acid sequence of phe pro phe pro phe pro phe pro phe pro (SEQ ID No. 86). This is identical to the amino acid sequence expressed by the +1 mutation. (Note that n=a nucleotide from the adjacent 5′ site [or an inserted nucleotide] and y=a nucleotide from the adjacent 3′ site). The amino acid sequence is the same with each frameshift even though the nucleotide sequence differs. Both out-of-frame nucleotide sequences contain an even number of codons, in this example 10, while the in-frame sequence has 11 codons.

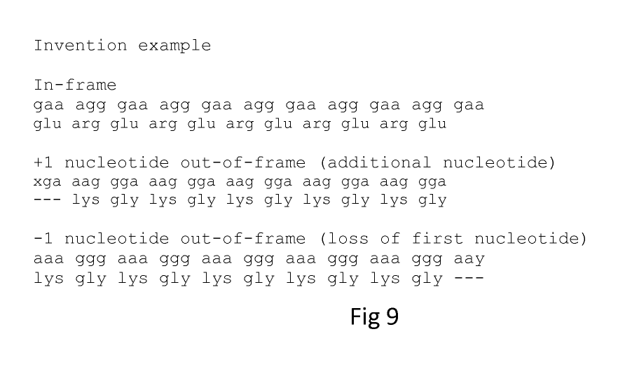

In FIG. 9, gaa agg gaa agg gaa agg gaa agg gaa agg gaa (SEQ ID No. 41) is 5 pairs of the codons gaa aag with an extra gaa codon added to the 3′ end. This sequence results in expression of the amino acid sequence glu arg glu arg glu arg glu arg glu arg glu (SEQ ID No. 127). A +1 nucleotide out-of-frame mutation (additional preceding nucleotide) gives a sequence of nga aag gga aag gga aag gga aag gga aag gga (SEQ ID No. 128) resulting in an amino acid sequence of - - - lys gly lys gly lys gly lys gly lys gly (SEQ ID No. 89). A −1 nucleotide out-of-frame mutation (loss of preceding nucleotide) gives a sequence of aaa ggg aaa ggg aaa ggg aaa ggg aaa ggg aay (SEQ ID No. 129) resulting in an amino acid sequence of lys gly lys gly lys gly lys gly lys gly (SEQ ID No. 89). This is identical to the sequence expressed by the +1 mutation. (Note that n=a nucleotide from the adjacent 5′ site [or an inserted nucleotide] and y=a nucleotide from the adjacent 3′ site). Both out-of-frame nucleotide sequences contain an even number of codons, in this example 10 codons, while the in-frame sequence has 11 codons.

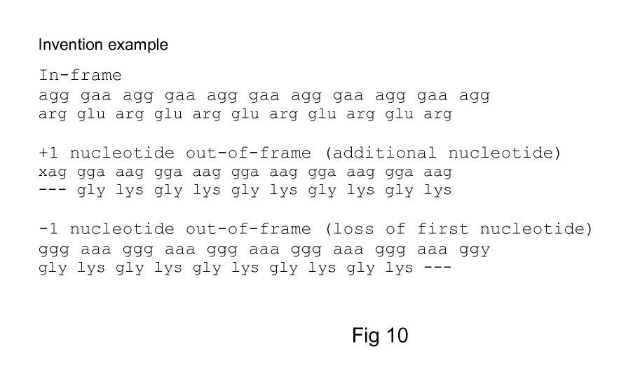

In FIG. 10, agg gaa agg gaa agg gaa agg gaa agg gaa agg (SEQ ID No. 40) is 5 pairs of the codons aag gaa with an extra agg codon added to the 3′ end. This sequence results in expression of the amino acid sequence arg glu arg glu arg glu arg glu arg glu arg (SEQ ID No. 84). A +1 nucleotide out-of-frame mutation (additional preceding nucleotide) gives a sequence of nag gga aag gga aag gga aag gga aag gga aag (SEQ ID No. 130) resulting in an amino acid sequence of gly lys gly lys gly lys gly lys gly lys (SEQ ID No. 131). A −1 nucleotide out-of-frame mutation (loss of preceding nucleotide) gives a sequence of ggg aaa ggg aaa ggg aaa ggg aaa ggg aaa ggy (SEQ ID No. 132) resulting in an amino acid sequence of gly lys gly lys gly lys gly lys gly lys (SEQ ID No. 131). This is identical to the sequence expressed by the +1 mutation. (Note that n=a nucleotide from the adjacent 5′ site [or an inserted nucleotide] and y=a nucleotide from the adjacent 3′ site). Both out-of-frame nucleotide sequences contain an even number of codons, in this example 10 codons, while the in-frame sequence has 11 codons.

It is useful to be able to detect amino acid sequences that result from in-frame expression and out-of-frame expression of nucleotide sequences of the present invention. Ligands can be made that specifically bind to selected amino acid sequences. Ligands can consist of antibodies, single chain antibodies, single domain antibodies artificial ligands and any other protein sequence that binds to a selected amino acid sequence. Ligands also can be made artificially from molecularly imprinted polymers or other polymers. Ligands can also be composed of other organic polymers such as RNA, DNA or peptide nucleic acids.

To increase avidity of ligand binding, multiple sets of affinity tags may be used, preferably connected by a linker to decrease possible steric hindrance. The linker preferably would consist of multiple repeats of ggg, aaa, ttt or ccc. Two, three, four or more repeats of 4-15 amino acids could be used. Linkers would not necessarily produce the same linker amino acid sequence from +1 and −1 frameshift mutations.

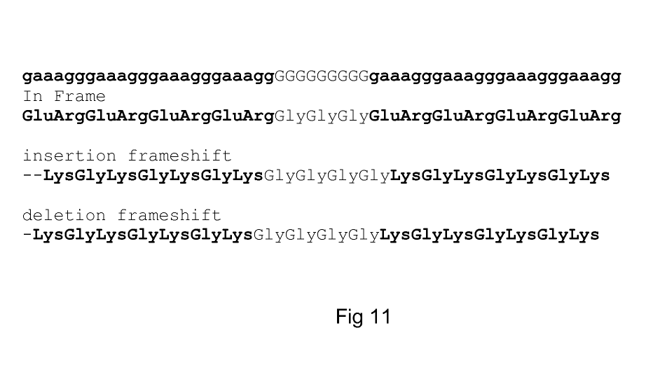

FIG. 11 shows an example where the linker is compatible with the sequence and forms similar linker amino acid sequences for all in-frame and out-of frame sequences. The sequence

(SEQ ID No. 133)
gaaagggaaagggaaagggaaaggGGGGGGGGGgaaagggaaagggaaa
gggaaagg

contains two sets of nucleotide sequences of the invention (in bold) connected by nine guanine nucleotides that encode three glycine amino acids when in-frame. The in-frame amino acid sequence is:

(SEQ ID No. 134)
GluArgGluArgGluArgGluArgGlyGlyGlyGluArgGluArgGlu
ArgGluArg.

The insertion (+1) frameshift amino acid sequence is:

(SEQ ID No. 135)
--LysGlyLysGlyLysGlyLysGlyGlyGlyGlyLysGlyLysGly
LysGlyLys

The deletion (−1) frameshift amino acid sequence is

(SEQ ID No. 135)
-LysGlyLysGlyLysGlyLysGlyGlyGlyGlyLysGlyLysGly
LysGlyLys.

The +1 and −1 frameshift amino acid sequences are the same. The linker is one that expresses three glycine amino acids in-frame and four glycine amino acids in each out-of-frame expression. Thus, the nucleotide sequence GGGGGGGGG works well as a linker.

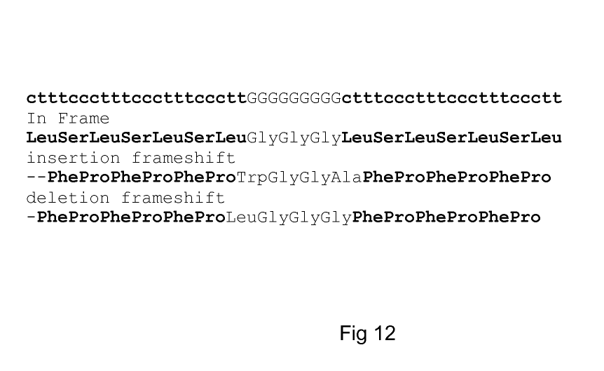

Other linkers produce results that differ among the expression frames. FIG. 12 shows an example of a linker that produces different linker results for the three expression frames while maintaining sequences compatible with the invention. The polynucleotide

(SEQ ID No. 136)
ctttccctttccctttcccttGGGGGGGGGctttccctttccctttccc
tt

expresses in-frame as

(SEQ ID No. 137)
LeuSerLeuSerLeuSerLeuGlyGlyGlyLeuSerLeuSerLeuSer
Leu

The insertion (+1) frameshift amino acid sequence is:

(SEQ ID No. 138)
--PheProPheProPheProTrpGlyGlyAlaPheProPheProPhe
Pro.

The deletion (−1) frameshift as amino acid sequence is:

(SEQ ID No. 139)
-PheProPheProPheProLeuGlyGlyGlyPheProPheProPhePro

FIG. 12 shows a linker sequence that produces variable amino acid sequences in the two out-of-frame directions while maintaining the correct sequences of the invention. This linker may be satisfactory, but it is less desirable than the linker shown in FIG. 11.

Another aspect of the invention are ligands, to include antibodies or antibody fragments, that specifically bind to an amino acid sequence encoded by a nucleotide sequence comprised of two or more repeats of nucleotide sequences selected from the list comprising ctttcc, tccctt, agggaa, gaaagg. The ligands would specifically bind to an amino acid sequence comprising two or more repeats of amino acid pairs selected from the list comprising proline-phenylalanine; phenylalanine-proline; lysine-glycine; glycine-lysine.

Another aspect of one embodiment of the invention are amino acid sequences selected from the list comprising (proline-phenylalanine)n+proline; (phenylalanine-proline)n+phenylalanine; (lysine-glycine)n+lysine; or (glycine-lysine)n+glycine where n represents n number of amino acid pairs where n is 2 or greater. This includes simple repeats of amino acid sequences containing two or more repeats of amino acid pairs selected from the list comprising proline-phenylalanine; phenylalanine-proline; lysine-glycine; glycine-lysine.

Another aspect of the invention is a nucleotide sequence and the amino acid sequence encoded by said nucleotide sequence wherein, said out-of-frame amino acid sequences are identical in all of the possible one or two out-of-frame reading frames.

One aspect of the invention are ligands that bind to amino acid sequences of the present invention; recognizing amino acid sequences that are comprised of repeats of the amino acid pairs proline-phenylalanine; phenylalanine-proline; lysine-glycine; or glycine-lysine.

Another aspect of the invention is ligands, to include antibodies and antibody fragments, that bind to amino acid sequences of the present invention. The ligands recognize amino acid sequences that are comprised of repeats of amino acids in the form of (proline-phenyalanine)n+proline; (phenylalanine-proline)n+phenylalanine; (lysine-glycine)n+lysine; (glycine-lysine)n+glycine; phenylalanine+(proline-phenylalanine)n; proline+(phenylalanine-proline)n; glycine+(lysine-glycine)n; or lysine+(glycine-lysine)n; (proline-phenyalanine)n; (phenylalanine-proline)n; (lysine-glycine)n; (glycine-lysine)n; where “n” is any integer greater than 1.

A person of ordinary skill in the art would recognize that antibody and antibody fragments can be obtained through the use of standard methods For example, antibodies can be made by vaccinating animals with peptides or proteins containing peptide sequences described herein. Hybridomas can be made using B cells collected from animals using immortalization techniques known to the art. For example, peptide specific, antibody secreting hybridomas can be made using electrofusion, polyethylene glycol fusion or other fusion methods to fuse antigen specific antibody secreting B cells with myeloma cells. Alternatively, antibodies can be made using molecular techniques. Libraries of antibody encoding genes can be isolated from vaccinated animals, unvaccinated animals or made synthetically. Antigen specific antibodies can be isolated from the antibody encoding DNA library using display techniques that maintain association of an expressed antibody and its encoding polynucleotide. A number of methods have been devised to identify protein-protein interactions that also allow for the recovery of genetic material that encodes the identified proteins. Some of these technologies work by reconstituting cellular functions in vivo while others utilize in vitro binding assays to identify physical interactions. Examples are the two-hybrid system, phage display, cellular display, ribosome display, and mRNA display.

It is recognized that traditional antibodies are only one form of ligand that can bind to a peptide sequence. Alternate protein scaffolds are known to the art. An example of one alternate scaffold is VHH fragments isolated from single domain camelid antibodies. Other scaffolds may be used to make ligands that bind the affinity TAGs described herein.

In one aspect of the invention, amino acid sequences of the present invention are amino acid sequences that are comprised of repeats of the amino acid pairs proline-phenylalanine; phenylalanine-proline; lysine-glycine; or glycine-lysine.

In another aspect of the invention, amino acid sequences of the present invention, are amino acid sequences that are comprised of repeats of amino acids in the form of (proline-phenylalanine)n+proline; (phenylalanine-proline)n+phenylalanine; (lysine-glycine)n+lysine; (glycine-lysine)n+glycine; phenylalanine+(proline-phenylalanine)n; proline+(phenylalanine-proline)n; glycine+(lysine-glycine)n or lysine+(glycine-lysine)n, where “n” is any integer greater than 1.

One form of the invention includes a nucleotide sequence that encodes one amino acid sequence when expressed in-frame and a different amino acid sequence when expressed out-of-frame as a result of a single nucleotide change, wherein said out-of-frame amino acid sequence is the same whether single nucleotide change is a nucleotide addition or deletion. This can comprise at least two repeats of the nucleotide sequence ctttcc (example SEQ ID No. 1, 9, 17, 25, 34, 42), at least two repeats of the nucleotide sequence tccctt (example SEQ ID No. 2, 10, 18, 26, 35, 43, 116), at least two repeats of the nucleotide sequence agggaa (example SEQ ID No. 3, 11, 19, 27, 36, 44), or at least two repeats of the nucleotide sequence gaaagg (example SEQ ID No. 4, 12, 20, 28, 29, 37, 45). A preferred embodiment would have 2-10 repeats of a nucleotide sequence selected from the list; gaaagg, cttcc, tcctt, or agggaa.

One form of the invention includes at least two repeats of the nucleotide sequence ctttcc followed by an additional ctt (example SEQ ID No. 5, 13, 21, 38, 46, 106). Another form includes at least two repeats of the nucleotide sequence tccctt followed by an additional tcc (example SEQ ID No. 6, 14, 22, 39, 47, 107). Another form includes at least two repeats of the nucleotide sequence agggaa followed by an additional agg (example SEQ ID No. 7, 15, 23, 40, 48, 108). Still another form includes at least two repeats of the nucleotide sequence gaaagg followed by and additional gaa (example SEQ ID No. 8, 16, 24, 41, 49, 109).

One form of the invention includes at least two repeats of the nucleotide sequence ctttcc preceded by an additional tcc (example SEQ ID No. 6, 14, 22, 39, 47, 107). Another form includes at least two repeats of the nucleotide sequence tccctt preceded by an additional ctt (example SEQ ID No. 5, 13, 21, 38, 46, 106). Another form includes at least two repeats of the nucleotide sequence agggaa preceded by an additional gaa (example SEQ ID No. 8, 16, 24, 41, 49, 109). Still another form includes at least two repeats of the nucleotide sequence gaaagg preceded by an additional agg (example SEQ ID No. 7, 15, 23, 40, 48, 108).

Another form of the invention includes a ligand that specifically binds to an amino acid sequence comprising two or more repeats of amino acid pairs selected from the list comprising in-frame amino acid pairs leucine-serine (example SEQ ID No. 30, 58, 90); serine-leucine (example SEQ ID No. 31, 59, 75, 91); arginine-glutamic acid (example SEQ ID No. 32, 60, 76, 92); glutamic acid-arginine (example SEQ ID No. 33, 61, 77, 93) or out-of-frame amino acid pairs proline-phenylalanine (example SEQ ID No. 55, 71, 87, 103); phenylalanine-proline (example SEQ ID No. 54, 70, 86, 102); lysine-glycine (example SEQ ID No. 57, 73, 89, 105); glycine-lysine (example SEQ ID No. 56, 72, 88, 104, 131). These amino acid sequences can have an additional amino acid in the form of in-frame amino acid pairs (leucine-serine)n+leucine (example SEQ ID No. 66, 74, 82, 98); (serine-leucine)n+serine (example SEQ ID No. 67, 83, 99); (arginine-glutamic acid)n+arginine (example SEQ ID No. 68, 84, 100); (glutamic acid-arginine)n+glutamic acid (example SEQ ID No. 69, 101, 127) or out-of-frame amino acid pairs (proline-phenylalanine)n+proline (example SEQ ID No. 51, 63, 79, 95); (phenylalanine-proline)n+phenylalanine (example SEQ ID No. 50, 62, 78, 94); (lysine-glycine)n+lysine (example SEQ ID No. 53, 65, 81, 85, 97); or (glycine-lysine)n+glycine (example SEQ ID No. 52, 64, 80, 96) wherein n represents n number of amino acid pairs and wherein n is 2 or greater and the total number of amino acids in the targeted sequence is an odd number.

Another form of the invention includes an amino acid sequence comprising two or more repeats of amino acid pairs selected from the list comprising in-frame amino acid pairs leucine-serine (example SEQ ID No. 30, 58, 90); serine-leucine (example SEQ ID No. 31, 59, 75, 91); arginine-glutamic acid (example SEQ ID No. 32, 60, 76, 92); glutamic acid-arginine (example SEQ ID No. 33, 61, 77, 93) or out-of-frame amino acid pairs proline-phenylalanine (example SEQ ID No. 55, 71, 87, 103); phenylalanine-proline (example SEQ ID No. 54, 70, 86, 102); lysine-glycine (example SEQ ID No. 57, 73, 89, 105); glycine-lysine (example SEQ ID No. 56, 72, 88, 104, 131). These sequences can additional an amino acid in the form of in-frame amino acid pairs (leucine-serine)n+leucine (example SEQ ID No. 66, 74, 82, 98); (serine-leucine)n+serine (example SEQ ID No. 67, 83, 99); (arginine-glutamic acid)n+arginine (example SEQ ID No. 68, 84, 100); (glutamic acid-arginine)n+glutamic acid (example SEQ ID No. 69, 101, 127) or out-of-frame amino acid pairs (proline-phenylalanine)n+proline (example SEQ ID No. 51, 63, 79, 95); (phenylalanine-proline)n+phenylalanine (example SEQ ID No. 50, 62, 78, 94); (lysine-glycine)n+lysine (example SEQ ID No. 53, 65, 81, 85, 97); or (glycine-lysine)n+glycine (example SEQ ID No. 52, 64, 80, 96) wherein n represents n number of amino acid pairs and wherein n is 2 or greater and the total number of amino acids in the sequence is an odd number.

Affinity TAGs describe herein may be used to purify proteins produced during in vivo or in vitro protein production. An antibody recognizing an affinity TAG can be attached to beads in a column using standard bioconjugation techniques and a biological solution containing a desired protein run through the column to separate, purify and/or concentrate the desired protein. Proteins are eluted from the column using high salt or high or low pH solutions.

The affinity TAGs described herein are especially useful to help monitor the quality of gene expression. An affinity TAG DNA sequence applied to a sequence immediately after the Kozac sequence or after a start codon will help monitor initial protein expression by identifying out-of-frame expression at the beginning of the sequence. The DNA encoding the affinity TAG can be placed any place down- stream of the start codon to include the 3′ end of the gene to detect frameshifts in other areas of the sequence. It is recognized that insertions closer to the 3′ end increase the risk that a frameshift mutation may create a stop codon prior to the TAG sequence, thus insertions closer to the 5′ end are more likely to detect a frameshift using the TAG sequence. An affinity TAG described herein can be placed at each end of a protein to monitor frame-shifts between TAGs. In this instance, an antibody recognizing an in-frame sequence is used to capture the desired protein while an antibody recognizing the out-of-frame sequence is used to detect frameshift mutations between the two TAG sites.

Frameshift detection using sequences described herein may be especially useful for monitoring bioprocesses. Ligands or antibodies detecting in-frame and out-of-frame gene expression would be used in assays. Assays showing the relative amount of in-frame TAG sequence and out-of-frame TAG sequence would provide a quantitative measure of process stability or deterioration.

TAG sequences also may be used as a marker to identify specific sets of proteins.

The invention has been described with references to a preferred embodiment. While specific values, relationships, materials and steps have been set forth for purposes of describing concepts of the invention, it will be appreciated by persons skilled in the art that numerous variations and/or modifications may be made to the invention as shown in the specific embodiments without departing from the spirit or scope of the basic concepts and operating principles of the invention as broadly described. It should be recognized that, in the light of the above teachings, those skilled in the art can modify those specifics without departing from the invention taught herein. Having now fully set forth the preferred embodiments and certain modifications of the concept underlying the present invention, various other embodiments as well as certain variations and modifications of the embodiments herein shown and described will obviously occur to those skilled in the art upon becoming familiar with such underlying concept. It is intended to include all such modifications, alternatives and other embodiments insofar as they come within the scope of the appended claims or equivalents thereof. It should be understood, therefore, that the invention may be practiced otherwise than as specifically set forth herein. Consequently, the present embodiments are to be considered in all respects as illustrative and not restrictive.

### REFERENCES

The following references cited in the specification are hereby incorporated by reference in their entirety.

### References

R. T. Raines et al., The S-Tag Fusion System for Protein Purification. Methods Enzymol. 326, 362-367 (2000)
T. P. Hopp, K. S. Prickett et al. (1988) A short polypeptide marker sequence useful for recombinant protein identification and purification. Nature Biotechnology 6:1204-1210.
A. Einhauer and A. Jungbaeur (2001) The FLAG™ peptide, a versatile fusion tag for the purification of recombinant proteins. J. Biochem. Biophys. Methods 49:455-65.
Hochuli, E.; Bannwarth, W.; Dobai, H.; Gentz, R.; Snüber, D. (1988). “Genetic Approach to Facilitate Purification of Recombinant Proteins with a Novel Metal Chelate Adsorbent”. Bio/Technology 6 (11): 1321-1325
Hengen, P (1995). “Purification of His-Tag fusion proteins from Escherichia coli”. Trends in Biochemical Sciences 20 (7): 285-6
Wilson, David S.; Keefe, Anthony D.; Szostak, Jack W. (2001). “The use of mRNA display to select high-affinity protein-binding peptides”. Proceedings of the National Academy of Sciences 98 (7): 3750-5.
Keefe, Anthony D.; Wilson, David S.; Seelig, Burckhard; Szostak, Jack W. (2001). “One-Step Purification of Recombinant Proteins Using a Nanomolar-Affinity Streptavidin-Binding Peptide, the SBP-Tag”. Protein Expression and Purification 23 (3): 440-6
Zakeri, B. and Howarth, M. (2010). Spontaneous intermolecular amide bond formation between side chains for irreversible peptide targeting. J. Am. Chem. Soc. 132, 4526-4527
Kang, H. J., Coulibaly, F., Clow, F., Proft, T., and Baker, E. N. (2007). Stabilizing isopeptide bonds revealed in gram-positive bacterial pilus structure. Science 318, 1625-1628. Schmidt, Thomas G M; Skerra, Arne (2007). “The Strep-tag system for one-step purification and high-affinity detection or capturing of proteins”. Nature Protocols 2 (6): 1528-35
Skerra, A; Schmidt, T G (2000). “Use of the Strep-Tag and streptavidin for detection and purification of recombinant proteins”. Methods in enzymology. Methods in Enzymology 326: 271-304.
U.S. Pat. No. 4,703,004
U.S. Pat. No. 5,872,209
U.S. Pat. No. 6,811,970
U.S. Pat. No. 6,846,804
U.S. Pat. No. 7,026,141
U.S. Pat. No. 7,070,941
U.S. Pat. No. 7,361,461
U.S. Pat. No. 7,473,535
U.S. Pat. No. 7,507,412
U.S. Pat. No. 7,745,163
U.S. Pat. No. 8,008,032
U.S. Pat. No. 8,178,316

What is claimed is:

1. A nucleotide sequence that encodes one amino acid sequence when expressed in-frame and a different amino acid sequence when expressed out-of-frame as a result of a single nucleotide change, wherein said out-of-frame amino acid sequence is the same whether the single nucleotide change is a nucleotide addition or deletion.

2. The nucleotide sequence of claim 1 comprising at least two repeats of the nucleotide sequence selected from the group consisting of ctttcc, tccctt, agggaa, and gaaagg.

3. The nucleotide sequence of claim 1 comprising at least one of the nucleotide sequences selected from the group consisting of SEQ ID No. 2, 10, 18, 26, 35, 43, 116, 1, 9, 17, 25, 34, 42, 3, 11, 19, 27, 36, 44, 4, 12, 20, 28, 29, 37, and 45.

4. The nucleotide sequence of claim 1 comprising 2-10 repeats of a nucleotide sequence selected from the group consisting of gaaagg, cttcc, tcctt, and agggaa.

5. The nucleotide sequence of claim 1 comprising at least two repeats of the nucleotide sequence ctttcc and an additional ctt following the at least two repeats.

6. The nucleotide sequence of claim 1 comprising at least two repeats of the nucleotide sequence tccctt and an additional tcc following the at least two repeats.

7. The nucleotide sequence of claim 1 comprising at least two repeats of the nucleotide sequence agggaa and an additional agg following the at least two repeats.

8. The nucleotide sequence of claim 1 comprising at least two repeats of the nucleotide sequence gaaagg and an additional gaa following the at least two repeats.

9. The nucleotide sequence of claim 1 comprising at least two repeats of the nucleotide sequence ctttcc and an additional tcc preceding the at least two repeats.

10. The nucleotide sequence of claim 1 comprising at least two repeats of the nucleotide sequence tccctt and an additional ctt preceding the at least two repeats.

11. The nucleotide sequence of claim 1 comprising at least two repeats of the nucleotide sequence agggaa and an additional gaa preceding the at least two repeats.

12. The nucleotide sequence of claim 1 comprising at least two repeats of the nucleotide sequence gaaagg and an additional agg preceding the at least two repeats.

13. A ligand that specifically binds to an amino acid sequence comprising at least two repeats of amino acid pairs selected from the group consisting of in-frame amino acid pairs leucine-serine (SEQ ID No. 30, 58, and 90); serine-leucine (SEQ ID No. 31, 59, 75, and 91); arginine-glutamic acid (SEQ ID No. 32, 60, 76, and 92); glutamic acid-arginine (SEQ ID No. 33, 61, 77, and 93) or out-of-frame amino acid pairs proline-phenylalanine (SEQ ID No. 55, 71, 87, and 103); phenylalanine-proline (SEQ ID No. 54, 70, 86, and 102); lysine-glycine (SEQ ID No. 57, 73, 89, and 105); glycine-lysine (SEQ ID No. 56, 72, 88, 104, and 131).

14. A ligand of claim 13 wherein the amino acid sequence further comprises an additional amino acid, such that the amino acid sequence is selected from the group consisting of in-frame amino acid pairs (leucine-serine)n+leucine (SEQ ID No. 66, 74, 82, 98); (serine-leucine)n+serine (SEQ ID No. 67, 83, 99); (arginine-glutamic acid)n+arginine (SEQ ID No. 68, 84, 100); (glutamic acid-arginine)n+glutamic acid (SEQ ID No. 69, 101, 127) or out-of-frame amino acid pairs (proline-phenylalanine)n+proline (SEQ ID No. 51, 63, 79, 95); (phenylalanine-proline)n+phenylalanine (SEQ ID No. 50, 62, 78, 94); (lysine-glycine)n+lysine (SEQ ID No. 53, 65, 81, 85, 97); or (glycine-lysine)n+glycine (SEQ ID No. 52, 64, 80, 96) wherein n represents n number of amino acid pairs and wherein n is 2 or greater and the total number of amino acids in the targeted sequence is an odd number.

15. An amino acid sequence comprising at least two repeats of amino acid pairs selected from the list consisting of in-frame amino acid pairs leucine-serine (example SEQ ID No. 30, 58, 90); serine-leucine (example SEQ ID No. 31, 59, 75, 91); arginine-glutamic acid (example SEQ ID No. 32, 60, 76, 92); glutamic acid-arginine (example SEQ ID No. 33, 61, 77, 93) or out-of-frame amino acid pairs proline-phenylalanine (example SEQ ID No. 55, 71, 87, 103); phenylalanine-proline (example SEQ ID No. 54, 70, 86, 102); lysine-glycine (example SEQ ID No. 57, 73, 89, 105); glycine-lysine (example SEQ ID No. 56, 72, 88, 104, 131).

16. An amino acid sequence of claim 15 wherein said amino acid sequence has an additional amino acid, such that said amino acid sequence is selected from the groupconsisting of in-frame amino acid pairs (leucine-serine)n+leucine (example SEQ ID No. 66, 74, 82, 98); (serine-leucine)n+serine (example SEQ ID No. 67, 83, 99); (arginine-glutamic acid)n+arginine (example SEQ ID No. 68, 84, 100); (glutamic acid-arginine)n+glutamic acid (example SEQ ID No. 69, 101, 127) or out-of-frame amino acid pairs (proline-phenylalanine)n+proline (example SEQ ID No. 51, 63, 79, 95); (phenylalanine-proline)n+phenylalanine (example SEQ ID No. 50, 62, 78, 94); (lysine-glycine)n+lysine (example SEQ ID No. 53, 65, 81, 85, 97); or (glycine-lysine)n+glycine (example SEQ ID No. 52, 64, 80, 96) wherein n represents n number of amino acid pairs and wherein n is 2 or greater and the total number of amino acids in the sequence is an odd number.


SEQUENCE LISTING (139 sequences)

SEQ ID NO: 1 - DNA, 12 bp, Artificial Sequence
  Synthetic Oligonucleotide
ctttccctttcc

SEQ ID NO: 2 - DNA, 12 bp, Artificial Sequence
  Synthetic Oligonucleotide
tccctttccctt

SEQ ID NO: 3 - DNA, 12 bp, Artificial Sequence
  Synthetic Oligonucleotide
agggaaagggaa

SEQ ID NO: 4 - DNA, 12 bp, Artificial Sequence
  Synthetic Oligonucleotide
gaaagggaaagg

SEQ ID NO: 5 - DNA, 15 bp, Artificial Sequence
  Synthetic Oligonucleotide
ctttccctttccctt

SEQ ID NO: 6 - DNA, 15 bp, Artificial Sequence
  Synthetic Oligonucleotide
tccctttccctttcc

SEQ ID NO: 7 - DNA, 15 bp, Artificial Sequence
  synthetic oligonucleotide
agggaaagggaaagg

SEQ ID NO: 8 - DNA, 15 bp, Artificial Sequence
  synthetic oligonucleotide
gaaagggaaagggaa

SEQ ID NO: 9 - DNA, 24 bp, Artificial Sequence
  synthetic oligonucleotide
ctttccctttccctttccctttcc

SEQ ID NO: 10 - DNA, 24 bp, Artificial Sequence
  synthetic oligonucleotide
tccctttccctttccctttccctt

SEQ ID NO: 11 - DNA, 24 bp, Artificial Sequence
  synthetic oligonucleotide
agggaaagggaaagggaaagggaa

SEQ ID NO: 12 - DNA, 24 bp, Artificial Sequence
  synthetic oligonucleotide
gaaagggaaagggaaagggaaagg

SEQ ID NO: 13 - DNA, 27 bp, Artificial Sequence
  synthetic oligonucleotide
ctttccctttccctttccctttccctt

SEQ ID NO: 14 - DNA, 27 bp, Artificial Sequence
  synthetic oligonucleotide
tccctttccctttccctttccctttcc

SEQ ID NO: 15 - DNA, 27 bp, Artificial Sequence
  synthetic oligonucleotide
agggaaagggaaagggaaagggaaagg

SEQ ID NO: 16 - DNA, 27 bp, Artificial Sequence
  synthetic oligonucleotide
gaaagggaaagggaaagggaaagggaa

SEQ ID NO: 17 - DNA, 18 bp, Artificial Sequence
  synthetic oligonucleotide
ctttccctttccctttcc

SEQ ID NO: 18 - DNA, 18 bp, Artificial Sequence
  synthetic oligonucleotide
tccctttccctttccctt

SEQ ID NO: 19 - DNA, 18 bp, Artificial Sequence
  synthetic oligonucleotide
agggaaagggaaagggaa

SEQ ID NO: 20 - DNA, 18 bp, Artificial Sequence
  synthetic oligonucleotide
gaaagggaaagggaaagg

SEQ ID NO: 21 - DNA, 21 bp, Artificial Sequence
  synthetic oligonucleotide
ctttccctttccctttccctt

SEQ ID NO: 22 - DNA, 21 bp, Artificial Sequence
  synthetic oligonucleotide
tccctttccctttccctttcc

SEQ ID NO: 23 - DNA, 21 bp, Artificial Sequence
  synthetic oligonucleotide
agggaaagggaaagggaaagg

SEQ ID NO: 24 - DNA, 21 bp, Artificial Sequence
  synthetic oligonucleotide
gaaagggaaagggaaagggaa

SEQ ID NO: 25 - DNA, 42 bp, Artificial Sequence
  synthetic oligonucleotide
ctttccctttccctttccctttccctttccctttccctttcc

SEQ ID NO: 26 - DNA, 42 bp, Artificial Sequence
  synthetic oligonucleotide
tccctttccctttccctttccctttccctttccctttccctt

SEQ ID NO: 27 - DNA, 42 bp, Artificial Sequence
  synthetic oligonucleotide
agggaaagggaaagggaaagggaaagggaaagggaaagggaa

SEQ ID NO: 28 - DNA, 42 bp, Artificial Sequence
  synthetic oligonucleotide
gaaagggaaagggaaagggaaagggaaagggaaagggaaagg

SEQ ID NO: 29 - DNA, 57 bp, Artificial Sequence
  synthetic oligonucleotide
gaaagggaaagggaaagggaaaggggggggggggaaagggaaagggaaagggaaagg

SEQ ID NO: 30 - protein, 14 aa, Artificial Sequence
  expressed peptide from synthetic oligonucleotide
LSLSLSLSLSLSLS

SEQ ID NO: 31 - protein, 14 aa, Artificial Sequence
  expressed peptide from synthetic oligonucleotide
SLSLSLSLSLSLSL

SEQ ID NO: 32 - protein, 14 aa, Artificial Sequence
  expressed peptide from synthetic oligonucleotide
RERERERERERERE

SEQ ID NO: 33 - protein, 14 aa, Artificial Sequence
  expressed peptide from synthetic oligonucleotide
ERERERERERERER

SEQ ID NO: 34 - DNA, 30 bp, Artificial Sequence
  synthetic oligonucleotide
ctttccctttccctttccctttccctttcc

SEQ ID NO: 35 - DNA, 30 bp, Artificial Sequence
  synthetic oligonucleotide
tccctttccctttccctttccctttccctt

SEQ ID NO: 36 - DNA, 30 bp, Artificial Sequence
  synthetic oligonucleotide
agggaaagggaaagggaaagggaaagggaa

SEQ ID NO: 37 - DNA, 30 bp, Artificial Sequence
  synthetic oligonucleotide
gaaagggaaagggaaagggaaagggaaagg

SEQ ID NO: 38 - DNA, 33 bp, Artificial Sequence
  synthetic oligonucleotide
ctttccctttccctttccctttccctttccctt

SEQ ID NO: 39 - DNA, 33 bp, Artificial Sequence
  synthetic oligonucleotide
tccctttccctttccctttccctttccctttcc

SEQ ID NO: 40 - DNA, 33 bp, Artificial Sequence
  synthetic oligonucleotide
agggaaagggaaagggaaagggaaagggaaagg

SEQ ID NO: 41 - DNA, 33 bp, Artificial Sequence
  synthetic oligonucleotide
gaaagggaaagggaaagggaaagggaaagggaa

SEQ ID NO: 42 - DNA, 36 bp, Artificial Sequence
  synthetic oligonucleotide
ctttccctttccctttccctttccctttccctttcc

SEQ ID NO: 43 - DNA, 36 bp, Artificial Sequence
  synthetic oligonucleotide
tccctttccctttccctttccctttccctttccctt

SEQ ID NO: 44 - DNA, 36 bp, Artificial Sequence
  synthetic oligonucleotide
agggaaagggaaagggaaagggaaagggaaagggaa

SEQ ID NO: 45 - DNA, 36 bp, Artificial Sequence
  synthetic oligonucleotide
gaaagggaaagggaaagggaaagggaaagggaaagg

SEQ ID NO: 46 - DNA, 39 bp, Artificial Sequence
  synthetic oligonucleotide
ctttccctttccctttccctttccctttccctttccctt

SEQ ID NO: 47 - DNA, 39 bp, Artificial Sequence
  synthetic oligonucleotide
tccctttccctttccctttccctttccctttccctttcc

SEQ ID NO: 48 - DNA, 39 bp, Artificial Sequence
  synthetic oligonucleotide
agggaaagggaaagggaaagggaaagggaaagggaaagg

SEQ ID NO: 49 - DNA, 39 bp, Artificial Sequence
  synthetic oligonucleotide
gaaagggaaagggaaagggaaagggaaagggaaagggaa

SEQ ID NO: 50 - protein, 13 aa, Artificial Sequence
  expressed peptide from synthetic oligonucleotide
FPFPFPFPFPFPF

SEQ ID NO: 51 - protein, 13 aa, Artificial Sequence
  expressed peptide from synthetic oligonucleotide
PFPFPFPFPFPFP

SEQ ID NO: 52 - protein, 13 aa, Artificial Sequence
  expressed peptide from synthetic oligonucleotide
GKGKGKGKGKGKG

SEQ ID NO: 53 - protein, 13 aa, Artificial Sequence
  expressed peptide from synthetic oligonucleotide
KGKGKGKGKGKGK

SEQ ID NO: 54 - protein, 14 aa, Artificial Sequence
  expressed peptide from synthetic oligonucleotide
FPFPFPFPFPFPFP

SEQ ID NO: 55 - protein, 14 aa, Artificial Sequence
  expressed peptide from synthetic oligonucleotide
PFPFPFPFPFPFPF

SEQ ID NO: 56 - protein, 14 aa, Artificial Sequence
  expressed peptide from synthetic oligonucleotide
GKGKGKGKGKGKGK

SEQ ID NO: 57 - protein, 14 aa, Artificial Sequence
  expressed peptide from synthetic oligonucleotide
KGKGKGKGKGKGKG

SEQ ID NO: 58 - protein, 8 aa, Artificial Sequence
  expressed peptide from synthetic oligonucleotide
LSLSLSLS

SEQ ID NO: 59 - protein, 8 aa, Artificial Sequence
  expressed peptide from synthetic oligonucleotide
SLSLSLSL

SEQ ID NO: 60 - protein, 8 aa, Artificial Sequence
  expressed peptide from synthetic oligonucleotide
RERERERE

SEQ ID NO: 61 - protein, 8 aa, Artificial Sequence
  expressed peptide from synthetic oligonucleotide
ERERERER

SEQ ID NO: 62 - protein, 7 aa, Artificial Sequence
  expressed peptide from synthetic oligonucleotide
FPFPFPF

SEQ ID NO: 63 - protein, 7 aa, Artificial Sequence
  expressed peptide from synthetic oligonucleotide
PFPFPFP

SEQ ID NO: 64 - protein, 7 aa, Artificial Sequence
  expressed peptide from synthetic oligonucleotide
GKGKGKG

SEQ ID NO: 65 - protein, 7 aa, Artificial Sequence
  expressed peptide from synthetic oligonucleotide
KGKGKGK

SEQ ID NO: 66 - protein, 9 aa, Artificial Sequence
  expressed peptide from synthetic oligonucleotide
LSLSLSLSL

SEQ ID NO: 67 - protein, 9 aa, Artificial Sequence
  expressed peptide from synthetic oligonucleotide
SLSLSLSLS

SEQ ID NO: 68 - protein, 9 aa, Artificial Sequence
  expressed peptide from synthetic oligonucleotide
RERERERER

SEQ ID NO: 69 - protein, 9 aa, Artificial Sequence
  expressed peptide from synthetic oligonucleotide
ERERERERE

SEQ ID NO: 70 - protein, 8 aa, Artificial Sequence
  expressed peptide from synthetic oligonucleotide
FPFPFPFP

SEQ ID NO: 71 - protein, 8 aa, Artificial Sequence
  expressed peptide from synthetic oligonucleotide
PFPFPFPF

SEQ ID NO: 72 - protein, 8 aa, Artificial Sequence
  expressed peptide from synthetic oligonucleotide
GKGKGKGK

SEQ ID NO: 73 - protein, 8 aa, Artificial Sequence
  expressed peptide from synthetic oligonucleotide
KGKGKGKG

SEQ ID NO: 74 - protein, 9 aa, Artificial Sequence
  synthetic peptide
LSLSLSLSL

SEQ ID NO: 75 - protein, 10 aa, Artificial Sequence
  synthetic peptide
SLSLSLSLSL

SEQ ID NO: 76 - protein, 10 aa, Artificial Sequence
  synthetic peptide
RERERERERE

SEQ ID NO: 77 - protein, 10 aa, Artificial Sequence
  synthetic peptide
ERERERERER

SEQ ID NO: 78 - protein, 9 aa, Artificial Sequence
  synthetic peptide
FPFPFPFPF

SEQ ID NO: 79 - protein, 9 aa, Artificial Sequence
  expressed peptide from synthetic oligonucleotide
PFPFPFPFP

SEQ ID NO: 80 - protein, 9 aa, Artificial Sequence
  expressed peptide from synthetic oligonucleotide
GKGKGKGKG

SEQ ID NO: 81 - protein, 9 aa, Artificial Sequence
  expressed peptide from synthetic oligonucleotide
KGKGKGKGK

SEQ ID NO: 82 - protein, 11 aa, Artificial Sequence
  expressed peptide from synthetic oligonucleotide
LSLSLSLSLSL

SEQ ID NO: 83 - protein, 11 aa, Artificial Sequence
  expressed peptide from synthetic oligonucleotide
SLSLSLSLSLS

SEQ ID NO: 84 - protein, 11 aa, Artificial Sequence
  expressed peptide from synthetic oligonucleotide
RERERERERER

SEQ ID NO: 85 - protein, 11 aa, Artificial Sequence
  expressed peptide from synthetic oligonucleotide
KGKGKGKGKGK

SEQ ID NO: 86 - protein, 10 aa, Artificial Sequence
  expressed peptide from synthetic oligonucleotide
FPFPFPFPFP

SEQ ID NO: 87 - protein, 10 aa, Artificial Sequence
  synthetic peptide
PFPFPFPFPF

SEQ ID NO: 88 - protein, 10 aa, Artificial Sequence
  expressed peptide from synthetic oligonucleotide
GKGKGKGKGK

SEQ ID NO: 89 - protein, 10 aa, Artificial Sequence
  expressed peptide from synthetic oligonucleotide
KGKGKGKGKG

SEQ ID NO: 90 - protein, 12 aa, Artificial Sequence
  expressed peptide from synthetic oligonucleotide
LSLSLSLSLSLS

SEQ ID NO: 91 - protein, 12 aa, Artificial Sequence
  expressed peptide from synthetic oligonucleotide
SLSLSLSLSLSL

SEQ ID NO: 92 - protein, 12 aa, Artificial Sequence
  expressed peptide from synthetic oligonucleotide
RERERERERERE

SEQ ID NO: 93 - protein, 12 aa, Artificial Sequence
  expressed peptide from synthetic oligonucleotide
ERERERERERER

SEQ ID NO: 94 - protein, 11 aa, Artificial Sequence
  expressed peptide from synthetic oligonucleotide
FPFPFPFPFPF

SEQ ID NO: 95 - protein, 11 aa, Artificial Sequence
  expressed peptide from synthetic oligonucleotide
PFPFPFPFPFP

SEQ ID NO: 96 - protein, 11 aa, Artificial Sequence
  expressed peptide from synthetic oligonucleotide
GKGKGKGKGKG

SEQ ID NO: 97 - protein, 11 aa, Artificial Sequence
  expressed peptide from synthetic oligonucleotide
KGKGKGKGKGK

SEQ ID NO: 98 - protein, 13 aa, Artificial Sequence
  expressed peptide from synthetic oligonucleotide
LSLSLSLSLSLSL

SEQ ID NO: 99 - protein, 13 aa, Artificial Sequence
  expressed peptide from synthetic oligonucleotide
SLSLSLSLSLSLS

SEQ ID NO: 100 - protein, 13 aa, Artificial Sequence
  expressed peptide from synthetic oligonucleotide
RERERERERERER

SEQ ID NO: 101 - protein, 13 aa, Artificial Sequence
  expressed peptide from synthetic oligonucleotide
ERERERERERERE

SEQ ID NO: 102 - protein, 12 aa, Artificial Sequence
  expressed peptide from synthetic oligonucleotide
FPFPFPFPFPFP

SEQ ID NO: 103 - protein, 12 aa, Artificial Sequence
  expressed peptide from synthetic oligonucleotide
PFPFPFPFPFPF

SEQ ID NO: 104 - protein, 12 aa, Artificial Sequence
  expressed peptide from synthetic oligonucleotide
GKGKGKGKGKGK

SEQ ID NO: 105 - protein, 12 aa, Artificial Sequence
  expressed peptide from synthetic oligonucleotide
KGKGKGKGKGKG

SEQ ID NO: 106 - DNA, 45 bp, Artificial Sequence
  synthetic oligonucleotide
ctttccctttccctttccctttccctttccctttccctttccctt

SEQ ID NO: 107 - DNA, 45 bp, Artificial Sequence
  synthetic oligonucleotide
tccctttccctttccctttccctttccctttccctttccctttcc

SEQ ID NO: 108 - DNA, 45 bp, Artificial Sequence
  synthetic oligonucleotide
agggaaagggaaagggaaagggaaagggaaagggaaagggaaagg

SEQ ID NO: 109 - DNA, 45 bp, Artificial Sequence
  synthetic oligonucleotide
gaaagggaaagggaaagggaaagggaaagggaaagggaaagggaa

SEQ ID NO: 110 - DNA, 45 bp, Homo sapiens
gacatccagttgacccagtctccatcctccctgtctgcatctgtg

SEQ ID NO: 111 - protein, 15 aa, Homo sapiens
DIQLTQSPSSLSASV

SEQ ID NO: 112 - protein, 10 aa, Homo sapiens
PSLHPPCLHL

SEQ ID NO: 113 - protein, 14 aa, Homo sapiens
HPVDPVSILPCICG

SEQ ID NO: 114 - DNA, 42 bp, Artificial Sequence
  synthetic oligonucleotide
nctttccctttccctttccctttccctttccctttccctttc

SEQ ID NO: 115 - DNA, 42 bp, Artificial Sequence
  synthetic oligonucleotide
tttccctttccctttccctttccctttccctttccctttccn

SEQ ID NO: 116 - DNA, 42 bp, Artificial Sequence; identical to SEQ ID NO: 26
  synthetic oligonucleotide

SEQ ID NO: 117 - DNA, 42 bp, Artificial Sequence
  synthetic oligonucleotide
ntccctttccctttccctttccctttccctttccctttccct

SEQ ID NO: 118 - DNA, 42 bp, Artificial Sequence
  synthetic oligonucleotide
ccctttccctttccctttccctttccctttccctttccctty

SEQ ID NO: 119 - DNA, 42 bp, Artificial Sequence
  synthetic oligonucleotide
nagggaaagggaaagggaaagggaaagggaaagggaaaggga

SEQ ID NO: 120 - DNA, 42 bp, Artificial Sequence
  synthetic oligonucleotide
gggaaagggaaagggaaagggaaagggaaagggaaagggaay

SEQ ID NO: 121 - DNA, 42 bp, Artificial Sequence
  synthetic oligonucleotide
ngaaagggaaagggaaagggaaagggaaagggaaagggaaag

SEQ ID NO: 122 - DNA, 42 bp, Artificial Sequence
  synthetic oligonucleotide
aaagggaaagggaaagggaaagggaaagggaaagggaaaggy

SEQ ID NO: 123 - DNA, 33 bp, Artificial Sequence
  synthetic oligonucleotide
ntccctttccctttccctttccctttccctttc

SEQ ID NO: 124 - DNA, 33 bp, Artificial Sequence
  synthetic oligonucleotide
ccctttccctttccctttccctttccctttccy

SEQ ID NO: 125 - DNA, 33 bp, Artificial Sequence
  synthetic oligonucleotide
nctttccctttccctttccctttccctttcctt

SEQ ID NO: 126 - DNA, 33 bp, Artificial Sequence
  synthetic oligonucleotide
tttccctttccctttccctttccctttccctty

SEQ ID NO: 127 - protein, 11 aa, Artificial Sequence
  expressed peptide from synthetic oligonucleotide
ERERERERERE

SEQ ID NO: 128 - DNA, 33 bp, Artificial Sequence
  synthetic oligonucleotide
ngaaagggaaagggaaagggaaagggaaaggga

SEQ ID NO: 129 - DNA, 33 bp, Artificial Sequence
  synthetic oligonucleotide
aaagggaaagggaaagggaaagggaaagggaay

SEQ ID NO: 130 - DNA, 33 bp, Artificial Sequence
  synthetic oligonucleotide
nagggaaagggaaagggaaagggaaagggaaag

SEQ ID NO: 131 - protein, 10 aa, Artificial Sequence
  expressed peptide from synthetic oligonucleotide
GKGKGKGKGK

SEQ ID NO: 132 - DNA, 33 bp, Artificial Sequence
  synthetic oligonucleotide
gggaaagggaaagggaaagggaaagggaaaggy

SEQ ID NO: 133 - DNA, 57 bp, Artificial Sequence; identical to SEQ ID NO: 29
  synthetic oligonucleotide

SEQ ID NO: 134 - protein, 19 aa, Artificial Sequence
  expressed peptide from synthetic oligonucleotide
ERERERERGGGERERERER

SEQ ID NO: 135 - protein, 18 aa, Artificial Sequence
  expressed peptide from synthetic oligonucleotide
KGKGKGKGGGGKGKGKGK

SEQ ID NO: 136 - DNA, 51 bp, Artificial Sequence
  synthetic oligonucleotide
ctttccctttccctttcccttgggggggggctttccctttccctttccctt

SEQ ID NO: 137 - protein, 17 aa, Artificial Sequence
  expressed peptide from synthetic oligonucleotide
LSLSLSLGGGLSLSLSL

SEQ ID NO: 138 - protein, 16 aa, Artificial Sequence
  expressed peptide from synthetic oligonucleotide
FPFPFPWGGAFPFPFP

SEQ ID NO: 139 - protein, 16 aa, Artificial Sequence
  expressed peptide from synthetic oligonucleotide
FPFPFPLGGGFPFPFP